(19) United States
(12) Patent Application Publication
(10) Pub. No.: US 2026/0224115 A1
(43) Pub. Date: Aug. 6, 2026

(54) METHOD, SYSTEM AND USES FOR DETERMINING ABDOMINAL AORTIC CALCIFICATION

(71) Applicant: Edith Cowan University, Joondalup, WA (AU)

(72) Inventors: John SCHOUSBOE, Bloomington, MN (US); David SUTER, Joondalup (AU); Naeha SHARIF, Joondalup (AU); Joshua LEWIS, Joondalup (AU); Syed Zulqarnain GILANI, Joondalup (AU)

(73) Assignee: Edith Cowan University, Joondalup, WA (AU)

(21) Appl. No.: 19/112,257

(22) PCT Filed: Dec. 28, 2022
(86) PCT No.: PCT/IB2022/000785

§ 371 Date: Mar. 16, 2025

Related U.S. Application Data
(60) Provisional application No. 63/434,589, filed on Dec. 22, 2022.

(30) Foreign Application Priority Data
Sep. 16, 2022 (AU) 2022902684

(51) Int. Cl. A61B 5/02; A61B 5/00; G06N 3/0455; G06N 3/0464; G06T 7/00

(52) U.S. Cl. CPC A61B 5/02007; A61B 5/4509; A61B 5/7267; G06N 3/0455; G06N 3/0464; G06T 7/0012; G06T 2207/20081; G06T 2207/20084; G06T 2207/30012; G06T 2207/30101

(57) ABSTRACT

Method, system and uses for determining abdominal aortic calcification involves determining abdominal aortic calcification from a lateral lumbar image and diagnostic/prognostic use thereof. A method of determining abdominal aortic calcification comprises receiving a lateral lumbar image, determining a score representing abdominal aortic calcification from the image using one or more processors, wherein the determining step comprises, encoding the image to identify visual features and decoding the visual features to compute a plurality of calcification scores, each for a segment of the abdominal aorta.

The present invention relates to determining abdominal aortic calcification from a lateral lumbar image and diagnostic/prognostic use thereof.

### BACKGROUND

The following discussion of the background art is intended to facilitate an understanding of the present invention only. It should be appreciated that the discussion is not an acknowledgement or admission that any of the material referred to was part of the common general knowledge as at the priority date of the application.

Cardiovascular Disease (CVD) is the leading cause of death globally, and a significant contributor to disability worldwide. Vascular calcification is a marker of asymptomatic CVD and occurs when calcium builds up within the walls of the arteries undergoing the atherosclerotic process. Calcification can often begin decades before clinical events, such as heart attacks or strokes, occur. The abdominal aorta is one of the first vascular beds where calcification is seen. The presence and extent of Abdominal Aortic Calcification (AAC) is associated with increased risk of future cardiovascular hospitalizations and death.

The extent and severity of AAC can be assessed using lateral-lumbar radiographs, lateral spine Vertebral Fracture Assessment (VFA), Dual-energy X-ray Absorptiometry (DXA) and Quantitative Computed Tomography (QCT). VFA and DXA scans have the least amount of radiation but are of lower resolution. These scans can be used to semi-quantify AAC using the widely adopted Kauppila 24-point scoring method (AAC24), which measures the calcification along the length of abdominal aorta from L1 to L4. However, acquiring manual assessments for DXA images is not only time-consuming and expensive but also subjective.

The AAC24 scoring system scores AAC relative to each vertebral height (L1 to L4) and is scored as; 0 (no calcification), 1 (≤⅓ of the aortic wall), 2 (>⅓ to ≤⅔ of the aortic wall) or 3 (>⅔ of the aortic wall) for both the anterior and posterior aortic walls giving a maximum possible score of 24.

Severity of AAC is typically categorized as: low (AAC24 score 0 or 1), moderate (AAC24 score 2-5) and high (AAC24 score 6 or greater).

Some preliminary work has been done to automatically predict an overall AAC24 score for radiographic scans using a machine learning model (Chaplin, L., Cootes, T.: Automated scoring of aortic calcification in vertebral fracture assessment images. In: Medical Imaging 2019: Computer-Aided Diagnosis. vol. 10950, pp. 811-819. SPIE (2019); Elmasri, K., Hicks, Y., Yang, X., Sun, X., Pettit, R., Evans, W.: Automatic detection and quantification of abdominal aortic calcification in dual energy x-ray absorptiometry. Procedia Computer Science 96, 1011-1021 (2016); and Reid, S., Schousboe, J. T., Kimelman, D., Monchka, B. A., Jozani, M. J., Leslie, W. D.: Machine learning for automated abdominal aortic calcification scoring of dxa vertebral fracture assessment images: A pilot study. Bone 148, 115943 (2021)). However, these techniques only produce an overall AAC24 score and have limited repeatable accuracy.

Further, human produced AAC24 scores are currently only used as an indicator of or prognosis of CVD.

The present invention has been developed in light of this background.

Throughout the specification unless the context requires otherwise, the word “comprise” or variations such as “comprises” or “comprising”, will be understood to imply the inclusion of a stated integer or group of integers but not the exclusion of any other integer or group of integers.

Throughout the specification unless the context requires otherwise, the word “include” or variations such as “includes” or “including”, will be understood to imply the inclusion of a stated integer or group of integers but not the exclusion of any other integer or group of integers.

### SUMMARY OF INVENTION

According to an aspect of the present invention there is provided a method of determining abdominal aortic calcification comprising:
receiving a lateral lumbar image that includes an abdominal aortic section of a patient's abdominal aorta, the abdominal aortic section including a plurality of abdominal aortic segments;
determining a score representing abdominal aortic calcification from the lateral lumbar image using one or more processors, wherein the determining step comprises:
   the one or more processors encoding the lateral lumbar image to identify visual features in the lateral lumbar image at the abdominal aortic section;
   the one or more processors implementing an anterior decoder that decodes the visual features to produce a plurality of anterior calcification scores, each anterior
calcification score associated with an anterior portion of one abdominal aortic segment; and
   the one or more processors implementing a posterior decoder that decodes the visual features to produce a plurality of posterior calcification scores, each posterior calcification score associated with a posterior portion of one abdominal aortic segment;
   wherein each of the anterior and posterior decoders is a trained machine learning decoder and the anterior decoder is trained separately to the posterior decoder.

In an embodiment, each abdominal aortic segment corresponds to one of L1, L2, L3 and L4 vertebrae of the patient.

In an embodiment, encoding comprises identifying L1, L2, L3 and L4 abdominal aortic segments in the lateral lumbar image.

In an embodiment, each of the anterior and posterior decoders comprises a CNN, and the method comprises training the anterior and posterior decoders using a long short-term memory to store a sequence of abdominal aortic segment calcification scores and an attention module.

In an embodiment, encoding comprises providing the lateral lumbar image to a trained convolutional neural network (CNN) and using the convolutional neural network (CNN) to extract visual features.

In an embodiment, visual features are obtained from a last convolutional layer of the convolutional neural network (CNN).

In an embodiment, the calcification scores are classified into risk categories.

In an embodiment, the risk categories are used to identify a prognosis of a disease.

In an embodiment, the risk categories comprise a risk of CVD.

In an embodiment, the method comprises comparing the risk of CVD to a threshold value and categorising the patient associated with the lateral lumbar image as having a risk of CVD when the threshold is exceeded.

In an embodiment, the risk categories comprise a risk of later life falls and/or fractures.

In an embodiment, the method comprises comparing the risk of later life falls and/or fractures to a threshold value and categorising the patient associated with the lateral lumbar image as having a risk of later life falls and/or fractures when the threshold is exceeded.

In an embodiment, the risk categories comprise a risk of later life osteoporosis or osteoporotic fracture.

In an embodiment, the method comprises comparing the risk of osteoporosis to a threshold value and categorising the patient associated with the lateral lumbar image as having a risk of osteoporosis when the threshold is exceeded.

In an embodiment, the risk categories comprise a risk of later life dementia.

In an embodiment, the method comprises comparing the risk of dementia to a threshold value and categorising the patient associated with the lateral lumbar image as having a risk of dementia when the threshold is exceeded.

In an embodiment, the risk categories comprise a risk of diabetes.

In an embodiment, the method comprises comparing the risk of diabetes to a threshold value and categorising the patient associated with the lateral lumbar image as having a risk of diabetes when the threshold is exceeded.

According to an aspect of the present invention there is provided a system for determining abdominal aortic calcification comprising:
at least one processor that determines a score representing abdominal aortic calcification from a received lateral lumbar image of a patient's abdominal aorta that includes an abdominal aortic section having a plurality of abdominal aortic segments;
the at least one processor:
   implementing an encoder that identifies visual features in the lateral lumbar image at the abdominal aortic section;
   implementing an anterior decoder to decode the visual features to produce a plurality of anterior calcification scores, each anterior calcification score associated with an anterior portion of one abdominal aortic segment; and
   implementing a posterior decoder to decode the visual features to produce a plurality of posterior calcification scores, each posterior calcification score associated with one abdominal aortic segment;
   wherein each of the anterior and posterior decoders is a trained machine learning decoder and the anterior decoder is trained separately to the posterior decoder.

In an embodiment, each abdominal aortic segment corresponds to one of L1, L2, L3 and L4 vertebrae of the patient.

In an embodiment, the encoder is arranged to identify L1, L2, L3 and L4 abdominal aortic segments in the lateral lumbar image.

In an embodiment, the system comprises an imager to produce the lateral lumbar image.

In an embodiment, the at least one processor is arranged to implement an image crop and the system comprises a resizer configured to crop and resize the lateral lumbar image into a predetermined size and resolution for encoding according to the source of the lateral lumbar image.

In an embodiment, the encoder comprises a trained convolutional neural network (CNN) to extract visual features from the lateral lumbar image. A last convolutional layer may output the visual features.

In an embodiment, each of the anterior and posterior decoders comprises a trained CNN. Each of the anterior and posterior decoders may comprise a long-short term memory network and an attention module.

In an embodiment, each of the anterior and posterior decoders comprises a global pooling layer, a dense layer with rectified linear activation units (Relu activation), and a dense layer with a linear activation.

In an embodiment, the at least one processor is arranged to implement a risk category classifier for determining a risk category for a disease.

According to an aspect of the present invention, there is provided a method of diagnosing a disease, the method comprising:
receiving a lateral lumbar image that includes an abdominal aortic section of a patient's abdominal aorta, the abdominal aortic section including a plurality of abdominal aortic segments;
determining a score representing abdominal aortic calcification from the lateral lumbar image using one or more processors, wherein the determining step comprises:
   the one or more processors encoding the lateral lumbar image to identify visual features in the image at the abdominal aortic segments;
   the one or more processors implementing an anterior decoder to decode the visual features to produce a plurality of anterior calcification scores, each anterior calcification score associated with an anterior portion of one abdominal aortic segment;
   the one or more processors implementing a posterior decoder to decode the visual features to produce a plurality of posterior calcification scores, each posterior calcification score associated with an posterior portion of one abdominal aortic segment;
   wherein each of the anterior and posterior decoders is a trained machine learning decoder and the anterior decoder is trained separately to the posterior decoder; and
the method comprising determining a risk category for a disease based on the determined plurality of anterior and posterior calcification scores.

In an embodiment the method comprises identifying a prognosis of the disease from the determined risk category.

Also disclosed is a use of abdominal aortic calcification determined by a method comprising:
receiving a lateral lumbar image;
determining a score representing abdominal aortic calcification from the image using one or more processors, wherein the determining step comprises:
encoding the image to identify visual features; decoding the visual features to compute a plurality of calcification scores, each for a segment of the abdominal aorta,
the use comprising determining a risk category for a disease based on the determined abdominal aortic calcification.

In an embodiment the use comprises identifying a prognosis of the disease from the determined risk category.

Also disclosed is a method of diagnosing or prognosing a disease or condition in a human comprising classifying into risk categories calcification scores obtained by a method of determining abdominal aortic calcification according to the invention as herein described.

In an embodiment of the method of diagnosing or prognosing a disease or health risk in a human, the disease comprises CVD, Diabetes, Dementia, or Osteoporosis.

In an embodiment of the method of diagnosing or prognosing a disease or health risk in a human, the health risk comprises a risk of later life falls and/or fractures.

Also disclosed is a screening test for diagnosing or prognosing a disease or condition in a human comprising classifying into risk categories calcification scores obtained by a method of determining abdominal aortic calcification according to the invention as herein described.

In an embodiment of the screening test for diagnosing or prognosing a disease or health risk in a human, the disease comprises CVD, Diabetes, Dementia, or Osteoporosis.

In an embodiment of the screening test for diagnosing or prognosing a disease or health risk in a human, the health risk comprises a risk of later life falls and/or fractures.

Also disclosed is a use of a method of determining abdominal aortic calcification according to the invention as herein described in the diagnosis or prognosis of a disease or health risk in a human.

In an embodiment of the use of a method of determining abdominal aortic calcification according to the invention as herein described, the disease comprises CVD, Diabetes, Dementia, or Osteoporosis.

In an embodiment of the use of a method of determining abdominal aortic calcification according to the invention as herein described, the health risk comprises a risk of later life falls and/or fractures.

Also disclosed is a method of treating a human with a disease comprising CVD, Diabetes, Dementia, or Osteoporosis, after diagnosing or prognosing the disease comprising classifying into risk categories calcification scores obtained by a method of determining abdominal aortic calcification according to the invention as herein described.

Also disclosed is a method of treating a human with a health risk that comprises a risk of later life falls and/or fractures, after diagnosing or prognosing the health risk comprising classifying into risk categories calcification scores obtained by a method of determining abdominal aortic calcification according to the invention as herein described.

Also disclosed is a program for controlling one or more processors comprising instructions stored in a non-volatile medium which control the processor to perform any one of the methods herein described or to operate as any one of the systems as herein described.

### BRIEF DESCRIPTION OF DRAWINGS

In order to provide a better understanding, embodiments of the present invention will be described, by way of example only, with reference to the accompanying drawings, in which:

FIG. 1 is a schematic diagram of a system for determining abdominal aortic calcification scores according to an embodiment of the present invention;

FIG. 2 is a schematic block diagram of a processor of the system of FIG. 1;

FIG. 3 is a schematic block diagram of a decoder of the processor of FIG. 2;

FIG. 4 is a schematic functional diagram of sub-decoders of the decoder of FIG. 3;

FIG. 5 is a schematic functional diagram of an attention module of each of the sub-decoders of FIG. 4;

FIG. 6 is a schematic diagram of a method of determining abdominal aortic calcification scores according to an embodiment of the present invention; and

FIG. 7 is a scatter plot of predicted accuracy of an example system according to an embodiment of the present invention and a scatter plot of predicted accuracy of another system for determining an abdominal aortic calcification score.

### DESCRIPTION OF EMBODIMENTS

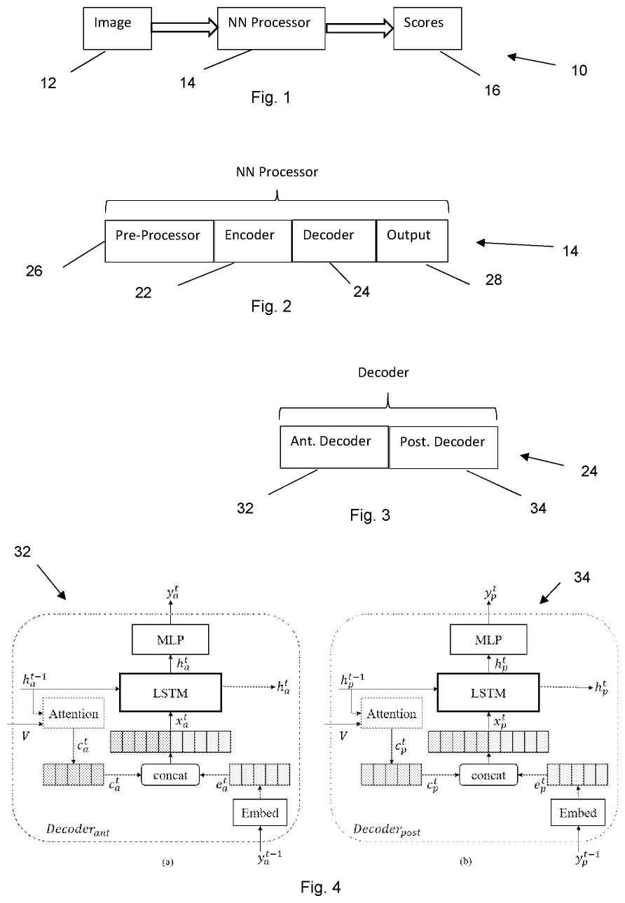

Referring to FIG. 1, there is shown a system 10 for determining abdominal aortic calcification scores 16 from a lateral lumbar image 12, the system comprising a neural network processor 14. The image 12 may be a lateral-lumbar radiograph, obtained from an imager machine for producing the lateral lumbar image. For example, the imager may be any of lateral spine Vertebral Fracture Assessment (VFA), Dual-energy X-ray Absorptiometry (DXA) or Quantitative Computed Tomography (QCT) machines. Other image sources may be used such as standard x-ray images and bone density images taken from a point of view that includes the relevant lumbar segments. In this embodiment, the image 12 needs to show a length of the abdominal aortic adjacent to each lumbar segment of the L1, L2, L3 and L4 vertebra in a human. Other aortic segments (including adjacent to a lower lumbar region) may be used in other mammals that are subject calcification. The scores 16 are able to be used to diagnose or prognose a disease or health risk in a human. The disease may comprise one or more of CVD, Diabetes, Dementia, or Osteoporosis. CVD comprises heart disease, stroke, and vascular diseases, of various types.

Referring to FIG. 2, the neural network processor 14 comprises a first subprocessor module operating as an encoder 22 for identifying visual features in the image 12 and a second sub-processor module operating as a decoder 24 for computing the calcification scores, each score for a segment of the abdominal aorta adjacent to each lumbar segment.

Preferably the image 12 is pre-processed by a pre-processor 26 before the encoder 22 identifies the visual features. Preferably the computed calcification scores from the decoder 24 are provided to a user by an output 28. Preferably the output 28 combines the computed the calcification scores into an overall score for output to the user. Preferably the output 28 applies an analysis to the computed the calcification scores and/or the overall score and provides the analysis to the user.

In an embodiment the pre-processor 26 is configured to crop and resize the image 12 according to the source of the image into a predetermined size and resolution for encoding. In an embodiment the pre-processor 26 comprises an image cropper and an image resizer. In an embodiment the cropping performed by the image cropper comprises applying one or more affine transformations. In an embodiment the resizing performed by the image resizer uses nearest neighbour interpolation.

In an embodiment the encoder 22 comprises a trained convolutional neural network (CNN) for extracting visual feature maps from the (preferably cropped and resized) image. In an embodiment the CNN is a Residual Network comprising a Deep Neural Network with Residual Blocks. In the Residual Blocks, a direct connection skips some layers of the neural network. In an embodiment a last convolutional layer, and not a subsequent classification layer of the CNN, outputs the visual feature maps. In an embodiment the CNN is trained using stochastic gradient descent with lateral-lumbar images and corresponding identified segments of the abdominal aorta in those images. The classification layer in the trained CNN would output the identification of the segments in each image but in this embodiment is only used for training purposes.

Referring to FIG. 3, in an embodiment the decoder 24 comprises at least two subdecoders, preferably an anterior decoder 32 for decoding the anterior wall of the abdominal aorta as anterior calcification scores adjacent to each of the lumbar segments, and a posterior decoder 34 for decoding the posterior wall of the abdominal aorta as anterior calcification scores adjacent to each of the lumbar segments. In an embodiment the sub-decoders (anterior decoder 32 and the posterior decoder 34) are independently trained classifiers.

Referring to FIG. 4, in an embodiment each sub-decoder 32, 34 comprises a trained neural network. In an embodiment each sub-decoder 32, 34 is trained using a long short-term memory network for storing a sequence of segmented scores and an attention module which produces the segment scores of the respective sub-decoder 32, 34.

In an embodiment each decoder comprises a global pooling layer, followed by a dense layer with rectified linear activation units (Relu activation), and another dense layer with a linear activation.

Each sub-decoder (a) and (b) in FIG. 4 independently maximizes the log likelihood over the parameter space:

$$\begin{matrix}{θ}^{*} = arg & \begin{matrix}max \\ θ\end{matrix} \sum ( V , y ) log p ( y \vert V ; θ )\end{matrix} \qquad ( 1 )$$

where θ represents the model parameters, V=[v1, v2, . . . , vn] represents the visual feature maps extracted from the pre-processed image, and y={y¹, y², . . . yⁿ} is the sequence of segmented scores.

The log-likelihood of the joint probability distribution log p(y|V; θ) can be decomposed as:

$$log p ( y \vert V ; θ ) = {\sum }_{( t = 1 )}^{T} log p ( {y}^{t} \vert {y}^{1} , … , {y}^{t - 1} , V ; θ ) \qquad ( 2 )$$

The Long-Short Term Memory (LSTM) module generates y, therefore the conditional probability log p(y|V) (dropping θ for convenience) can be modelled as log p(y^{t}|y¹, . . . y^{t-1}, V)=g(h^{t}, c^{t}) where g is a nonlinear function, c^{t} is the context vector and h^{t} is the hidden state of the LSTM at time t. h^{t} is modelled as h^{t}=LSTM (s^{t}, h^{t-1}, m^{t-1}) where s^{t} is the input vector, and h^{t-1} and m^{t-1} are hidden state and memory cell vectors at time t−1, respectively.

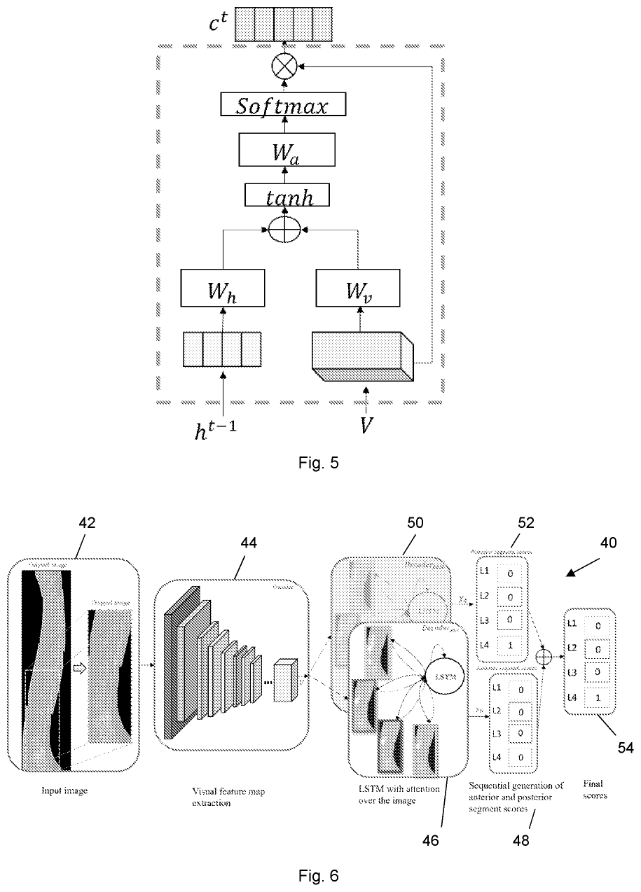

Referring to FIG. 5, to compute the context vector c^{t} an attention module is used, such that the context is dependent on specific regions in the image (via image feature maps) as well as the sub-decoder outputs. Therefore, c^{t} can be defined as c^{t}=q(V, h^{t-1}) where q is the attention function, and h^{t} is the hidden state of the LSTM at time t. The distribution of attention over the feature maps V (corresponding to various regions of the image) is computed using a feed-forward network and can be formalized as z^{t}=W^{α} tan h (W^{v}V+W^{h}h^{t-1}) and β^{t}=softmax(z^{t}) where W^{a}, W^{v} and W^{h} are the learnable parameters, and β is the attention weight over the feature maps V. Finally, c^{t} can be computed as:

$${c}^{t} = \sum \begin{matrix}n \\ i = \\ 1\end{matrix} {β}^{ti} {v}^{ti} \qquad ( 3 )$$

The model is trained using weighted cross-entropy loss, where the weights for each class are set based on the data distribution.

The two sub-decoders are trained independently to maximize the objective function given in Equation 2.

In an embodiment the output 28 is configured to combine each of the anterior L1, L2, L3 and L4 scores into a combined anterior score and to combine each of the posterior L1, L2, L3 and L4 scores into a combined posterior score. In an embodiment the output is configured to combining the combined anterior score and the combined posterior score into an overall score.

In an alternative embodiment the output 28 is configured to combine each of the anterior and posterior calcification scores for each of L1, L2, L3 and L4 into combined L1, L2, L3 and L4 scores. In an embodiment the method comprises combining each of the combined L1, L2, L3 and L4 scores into an overall score.

A method of use and operation of the system 10 to determine abdominal aortic calcification is now described with reference to FIG. 6.

A lateral lumbar image 12 is received at 42 from an imager machine. The processor 14 determines the score 16 representing abdominal aortic calcification from the image 12. The pre-processor 26 crops and resizes the image into a predetermined size and resolution for encoding according to the source of the image.

At 44 the encoder 22 extracts visual feature maps. At 46 and 50 each sub-decoder 32, 34 decodes the visual feature maps into a set of calcification scores is a set of anterior calcification scores 48 and a set of posterior calcification scores 52. The output 28 provides 54 the combined L1 to L4 set of scores 12. The output 28 may also provide a combined AAC24 score from the combined L1 to L4 set of scores 12.

In an embodiment the output 28 is configured to classify the combined L1 to L4 set of scores 12 (or the combined AAC24 score) into risk categories.

In an embodiment the output 28 is configured to classify the risk categories to identify a prognosis of a disease.

In an embodiment the risk categories comprise a risk of CVD. In an embodiment the method comprises comparing the risk of CVD to a threshold value and when the threshold is exceeded the patient the subject of the image is categorised as having a risk of CVD. A categorisation of risk of CVD can be used as a screening tool for referral to a cardiologist.

In an embodiment the risk scores comprise a risk of later life falls and/or fractures. In an embodiment the method comprises comparing the risk of later life falls and/or fractures to a threshold value and when the threshold is exceeded the patient the subject of the image is categorised as having a risk of later life falls and/or fractures. A categorisation of risk of later life falls and/or fractures can be used as a screening tool for referral for intervention.

In an embodiment the risk scores comprise a risk of later life Osteoporosis. In an embodiment the method comprises comparing the risk of Osteoporosis to a threshold value and when the threshold is exceeded the patient the subject of the image is categorised as having a risk of Osteoporosis. A categorisation of risk of later life Osteoporosis can be used as a screening tool for referral for intervention.

In an embodiment the risk scores comprise a risk of later life Dementia. In an embodiment the method comprises comparing the risk of Dementia to a threshold value and when the threshold is exceeded the patient the subject of the image is categorised as having a risk of Dementia. A categorisation of risk of later life Dementia can be used as a screening tool for referral for intervention.

In an embodiment the risk scores comprise a risk of later life Diabetes. In an embodiment the method comprises comparing the risk of Diabetes to a threshold value and when the threshold is exceeded the patient the subject of the image is categorised as having a risk of Diabetes. A categorisation of risk of later life Diabetes can be used as a screening tool for referral for intervention and/or medication and/or dietary changes and/or lifestyle changes.

### EXAMPLE

A dataset is comprised of randomly selected 1,916 bone-density machine derived lateral-spine scans, obtained using iDXA GE machines with a resolution of at least 1600×300 pixels was used to train the system. The disease severity distribution of the 1,916 scans was: low risk 829, moderate risk 445 and high risk 642. Although, these scans come with expert annotated AAC24 scores, the location of calcified pixels was not annotated on the scans. The data set had a distribution of zero scores is highly skewed for L1 and L2 perhaps because vascular calcification usually starts around L4 and L3 and then progresses upwards. Anterior segments in the data set had 176 unique (out of 44=256 possible) combinations but only 29 of them appeared more than 10 times. The most frequent sequence was [0,0,0,0], which appeared 904 times followed by [0,0,0,1], which appeared 77 times. For posterior segments, the dataset had 190 unique combinations, out of which only 30 appeared more than 10 times. Once again, [0,0,0,0] was the most frequent combination and appeared 786 (41%) times.

The distribution of scores in the dataset was as follows:

Score for each segment

| Segment | 0 | 1 | 2 | 3 |
|---|---|---|---|---|
| Anterior | 5390 | 1255 | 518 | 501 |
| Posterior | 5098 | 1251 | 685 | 630 |

The pre-processor cropped 50% from the top, 40% from the left and 10% from the right side of each scan. The cropped images were resized to 900×300 pixels using the nearest neighbour interpolation, and re-scaled to values between 0 and 1.

The training dataset was augment by applying various affine transformations to the images, such as translation [+20, −20], scaling [+20, −20], shear [0.01°, 0.05°] and rotation [+10°, −10]. The TorchVision library for data augmentation and the PyTorch Machine Learning Library were used for model training and evaluation.

A Resnet 152v2 was pretrained on ImageNet to be used as the encoder 22, although other models may be used. Feature maps from the last convolutional layer without using the classification layer of the pre-trained CNN. For an input image size of 900×300, the size of the extracted feature map is 29×10×2048. The feature maps are flattened to 290×2048 and feed them individually to the two decoding networks, which are termed Decoderant, and Decoderpost.

The two decoders, Decoderant, and Decoderpost, were trained independently with sequences of anterior and posterior segment ground truth scores, respectively. Furthermore, after training was complete, the output scores of both decoders (for a given test image) are summed to get a single score corresponding to each lumbar vertebrae. Finally, the scores of L1-L4 are summed to obtain the AAC24 scores.

Both decoders were comprised of an LSTM, with a hidden size of 512, and based on an attention module, where the output sequence length is 4. 10-fold stratified cross validation was performed (where the data is split based on the distribution of AAC24 scores, such that this distribution is maintained across all splits). In each fold 1,724 examples are used to train the network and 192 for validation. Early stopping was performed based on the average Pearson correlation between the predicted and ground truth segment scores. Dropout (first after the hidden layer of LSTM (alpha=0.5), then another (alpha=0.4) was used before the last FC layer) as a regularization strategy.

The resulting scores were evaluated by summing of all individual granular scores using the same dataset. In comparison to the human assessments, classifying patients into the three risk categories of low, medium and high, had an accuracy, sensitivity, and specificity of 82%, 74% and 80% respectively on the test set. The AAC24 scores generated by the present invention were highly correlated (>80%) with human assessments.

The Reid et al. pipeline model (referred to above in the background) was implemented (as M_{base}) (with minor modifications) to compare with results from the system 10 (referred to as M_{fgs}). In M_{base} a baseline CNN was trained with Resnet 152v2 as its encoder. The decoder consists of a global pooling layer, followed by a dense layer with Relu activation, and another dense layer with a linear activation. The generated AAC24 scores are classified into three risk levels, based on the risk thresholds.

Performance comparison of the M_{fgs} model with the baseline M_{base} (NPV is Negative Predictive Value and PPV is Positive Predictive Value) in one-vs-rest setting using the cumulative AAC24 predicted scores, follows.

| | Low (n = 829) | | Moderate (n = 445) | | High (n = 642) | | Mean | |
|---|---|---|---|---|---|---|---|---|
| | Mbase | Mfgs | Mbase | Mfgs | Mbase | Mfgs | Mbase | Mfgs |
| Accuracy | 71.14 | 82.52 | 62.06 | 75.52 | 79.12 | 87.89 | 70.77 | 81.98 |
| Sensitivity | 55.49 | 86.37 | 59.33 | 37.53 | 54.83 | 80.22 | 56.55 | 68.04 |
| Specificity | 83.07 | 79.58 | 62.88 | 87.02 | 91.37 | 91.76 | 79.11 | 86.12 |
| NPV | 70.99 | 88.45 | 83.63 | 82.16 | 80.06 | 90.20 | 78.23 | 86.93 |
| PPV | 71.43 | 76.33 | 32.59 | 46.65 | 76.19 | 83.06 | 60.07 | 68.68 |

M_{fgs} average classification accuracy 81.98+/−2.5% is significantly better than the baseline accuracy of 70.77+/−3.2%. Similarly, M_{fgs} average 3-class classification accuracy is 72.8+/−2.9% while that of the baseline is 55.8+/−3.2%. Accordingly, the M_{fgs} model predicts AAC24 scores more accurately compared to the baseline model.

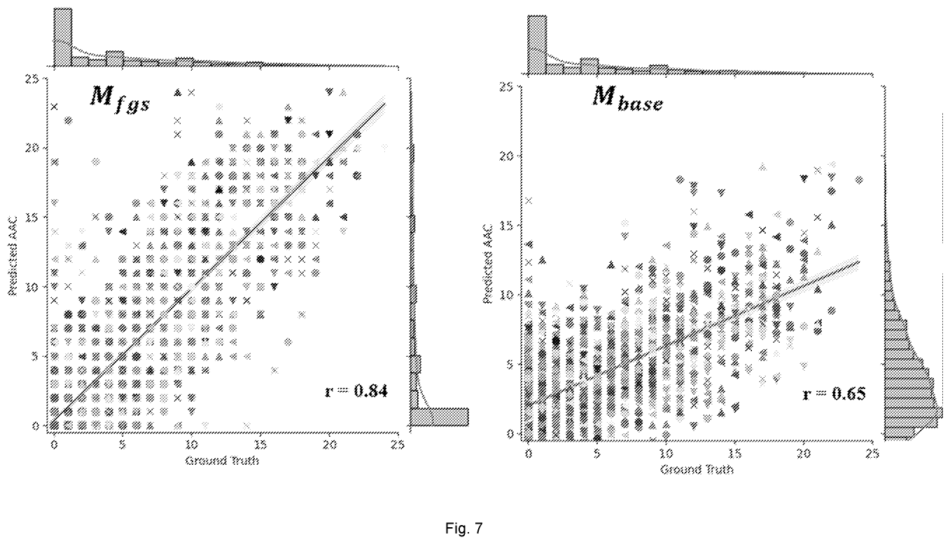

The output of a single AAC24 score (for all lumbar regions) from M_{fgs} is compared with the corresponding ground truth scores in the scatter plots of FIG. 7 and confusion matrix in the tables below. The M_{fgs} model is very good at classifying low and high risk patients. The figure provides evidence that fine-grained scoring results in significantly (p<<0.01) better prediction and higher correlation with human-scores.

| Ground Truth | Low | Moderate | High |
|---|---|---|---|
| | Predicted (M_{fgs}) | | |
| Low | 716 | 85 | 28 |
| Moderate | 201 | 167 | 77 |
| High | 21 | 106 | 515 |
| | Predicted (M_{base}) | | |
| Low | 460 | 308 | 61 |
| Moderate | 132 | 264 | 49 |
| High | 52 | 238 | 352 |

It was also ascertained whether predicting AAC scores in two segments is better than predicting them horizontally across each lumbar region e.g. L1 or L2 by training a variant of the model with a single decoder to predict a sequence of scores for each lumbar vertebra, L1L4, where the score for L1, would be the sum of L1ₐₙₜ and L1ₚₒₛₜ.

The variant model (M·_{fgs}) was used to determine whether predicting AAC scores in two segments is better than predicting them horizontally across each lumbar region e.g. L1 or L2. M·_{fgs} was trained as a variant of the M_{fgs} model with a single decoder to predict a sequence of scores for each lumbar vertebra, L1-L4, where the score for L1, would be the sum of L1ₐₙₜ and L1ₚₒₛₜ. A comparison between predicted AAC scores horizontally across each vertebrae vs predicting the scores vertically for each segment (anterior and posterior), i.e. comparison between M·_{fgs} and M_{fgs} is shown in the table below.

| | L1 | | L2 | | L3 | | L4 | |
|---|---|---|---|---|---|---|---|---|
| | ${M}_{fgs}^{*}$ | M_{fgs} | ${M}_{fgs}^{*}$ | M_{fgs} | ${M}_{fgs}^{*}$ | M_{fgs} | ${M}_{fgs}^{*}$ | M_{fgs} |
| Pearson | 0.40 | 0.49 | 0.56 | 0.64 | 0.56 | 0.70 | 0.64 | 0.69 |
| Correlation ↑ | | | | | | | | |
| Kendall | 0.34 | 0.44 | 0.49 | 0.56 | 0.47 | 0.60 | 0.54 | 0.58 |
| Correlation ↑ | | | | | | | | |
| Mean Absolute↑ | 0.96 | 0.60 | 0.73 | 0.67 | 1.20 | 0.88 | 1.13 | 1.10 |
| Error ↓ | | | | | | | | |

The correlation between human annotated scores of those predicted by M_{fgs} is significantly better (p<0.01) than the correlation produced by our variant M·_{fgs}.

### Use of the Scores

The output 28 of the system 10 may be configured to identify from the risk categories a risk of contracting or a predicted prognosis of a disease or condition. The predicted prognosis may be used as a screening tool for further investigation of the predicted prognosis by a relevant professional and/or to provide a remediation/prevention treatment or as a step in a diagnosis.

The risk scores for CVD are noted above. A preventative treatment of predicted prognosis of CVD comprises increase in consumption of fruit and vegetables, improved diet, reduce sitting time, and/or an increase in physical activity.

A risk score for osteoporosis can be determined from the AAC24 score, where for example the combined AAC24 score is inversely related to hip bone mineral density (rs=0.077, p=0.013) and heel broadband ultrasound attenuation (rs=−0.074, p=0.020) and stiffness index (rs=−0.073, p=0.022). Severe AAC is more likely to have prevalent fracture and lumbar spine injury. Moderate to severe AAC (AAC24 score>1) have increased fracture risk (HR 1.48 [1.15-1.91], p=0.002; HR 1.46 [1.07-1.99], p=0.019, respectively) compared to with low AAC.

A preventative treatment of predicted prognosis of osteoporosis comprises oral calcium supplements.

A risk score for fall-related hospitalizations can be determined from the AAC24 score due to weaker grip strength. In an embodiment the AAC24 score indicating risk of fall-related hospitalizations is at least 2, 3, 4, 5, or 6. In an embodiment there is a strong indication of risk of fall-related hospitalizations when the AAC24 score is at least 6, 7, 8, 9 or 10. In a study, over 14.5-years, 413 (39.2%) women experienced a fall-related hospitalization. Using a multivariable-adjusted model, each unit increase in baseline AAC24 was associated with a 3% increase in relative hazards for a fall-related hospitalization (HR 1.03 95% CI, 1.01 to 1.07). Compared to women with no AAC, women with any AAC had a 40% (HR 1.40 95% CI, 1.11 to 1.76) and 39% (HR 1.39 95% CI, 1.10 to 1.76) greater risk for fall-related hospitalizations in the minimal and multivariable-adjusted models, respectively.

Furthermore, where there is the presence of AAC more than 7 out of 10 women are associated with 39% higher risk for a fall-related hospitalization compared to women with no AAC.

A preventative treatment of predicted prognosis of fall-related hospitalizations comprise fall prevention programs including strengthening exercises.

A risk score for dementia can be determined from the AAC24 score. In an embodiment the AAC24 score indicating risk of dementia is at least 2, 3, 4, 5, or 6. In an embodiment there is a strong indication of risk of dementia when the AAC24 score is at least 6, 7, 8, 9 or 10. In a study of a baseline, women were 75.0+/−2.6 years, 44.7% had low AAC, 36.4% had moderate AAC and 18.9% had extensive AAC. Over 14.5-years, 150 (15.7%) women had a late-life dementia hospitalisation (n=132) and/or death (n=58). Compared to those with low AAC, women with moderate and extensive AAC were more likely to suffer latelife dementia hospitalisations (9.3%, 15.5%, 18.3%, respectively) and deaths (2.8%, 8.3%, 9.4%, respectively). After adjustment for cardiovascular risk factors and APOE, women with moderate and extensive AAC had twice the relative hazards of late-life dementia (moderate, aHR 2.03 95% CI 1.38-2.97; extensive, aHR 2.10 95% CI 1.33-3.32), compared to women with low AAC.

A preventative treatment of predicted prognosis of dementia comprises lifestyle modification and medication.

A risk score for diabetes can be determined from the AAC24 score. In an embodiment the diabetes is type I, alternatively, it is type II, or alternatively it is both type I and type II. In an embodiment the AAC24 score indicating risk of future diabetes is at least 2, 3, 4, 5, or 6. In an embodiment there is a strong indication of risk of future diabetes when the AAC24 score is at least 6, 7, 8, 9 or 10. In a study AAC was more prevalent in patients with diabetes mellitus (DM) with 29% AAC prevalence in DM (n=70) vs. 17% in non-DM men (n=62) (p=0.05), and 26% vs. 19% AAC prevalence in DM (n=63) vs. non-DM women (n=82) (p=0.06).

A preventative treatment of predicted prognosis of diabetes comprises lifestyle modification and medication.

The present invention not only overcomes the bottlenecks of manual AAC24 determination, but also provides improved results being sequential “fine-grained” scoring and a more accurate derived overall score. This can be used in diagnosis and/or prognosis of some diseases.

Modifications may be made to the present invention within the context of that described and shown in the drawings. Such modifications are intended to form part of the invention described in this specification.

1. A method of determining abdominal aortic calcification comprising:
receiving a lateral lumbar image that includes an abdominal aortic section of a patient's abdominal aorta, the abdominal aortic section including a plurality of abdominal aortic segments;
determining a score representing abdominal aortic calcification from the lateral lumbar image using one or more processors, wherein the determining step comprises:
the one or more processors encoding the lateral lumbar image to identify visual features in the lateral lumbar image at the abdominal aortic section;
the one or more processors implementing an anterior decoder that decodes the visual features to produce a plurality of anterior calcification scores, each anterior calcification score associated with an anterior portion of one abdominal aortic segment; and
the one or more processors implementing a posterior decoder that decodes the visual features to produce a plurality of posterior calcification scores, each posterior calcification score associated with a posterior portion of one abdominal aortic segment;
wherein each of the anterior and posterior decoders is a trained machine learning decoder and the anterior decoder is trained separately to the posterior decoder.

2. A method according to claim 1, wherein each abdominal aortic segment corresponds to one of L1, L2, L3 and L4 vertebrae of the patient, and encoding comprises identifying L1, L2, L3 and L4 abdominal aortic segments in the lateral lumbar image.

3. (canceled)

4. A method according to claim 1, wherein each of the anterior and posterior decoders comprises a CNN, and the method comprises training the anterior and posterior decoders using a long short-term memory to store a sequence of abdominal aortic segment calcification scores and an attention module.

5. A method according to claim 1, wherein encoding comprises providing the lateral lumbar image to a trained convolutional neural network (CNN) and using the convolutional neural network (CNN) to extract visual features.

6. A method according to claim 5, wherein the visual features are obtained from a last convolutional layer of the convolutional neural network (CNN).

7. A method according to claim 1, wherein the calcification scores are classified into risk categories.

8. A method according to claim 7, wherein the risk categories are used to identify a prognosis of a disease.

9. A method according to claim 7, wherein the risk categories comprise a risk of CVD, a risk of later life falls and/or fractures, a risk of later life osteoporosis or osteoporotic fracture, a risk of later life dementia, and/or a risk of diabetes.

10. (canceled)

11. (canceled)

12. (canceled)

13. (canceled)

14. (canceled)

15. (canceled)

16. (canceled)

17. (canceled)

18. (canceled)

19. A system for determining abdominal aortic calcification comprising:
at least one processor that determines a score representing abdominal aortic calcification from a received lateral lumbar image of a patient's abdominal aorta that includes an abdominal aortic section having a plurality of abdominal aortic segments;
the at least one processor:
implementing an encoder that identifies visual features in the lateral lumbar image at the abdominal aortic section;
implementing an anterior decoder to decode the visual features to produce a plurality of anterior calcification scores, each anterior calcification score associated with an anterior portion of one abdominal aortic segment; and
implementing a posterior decoder to decode the visual features to produce a plurality of posterior calcification scores, each posterior calcification score associated with one abdominal aortic segment;
wherein each of the anterior and posterior decoders is a trained machine learning decoder and the anterior decoder is trained separately to the posterior decoder.

20. A system according to claim 19, wherein each abdominal aortic segment corresponds to one of L1, L2, L3 and L4 vertebrae of the patient, and the encoder is arranged to identify L1, L2, L3 and L4 abdominal aortic segments in the lateral lumbar image.

21. (canceled)

22. (canceled)

23. (canceled)

24. A system according to claim 19, wherein the encoder comprises a trained convolutional neural network (CNN) to extract visual features from the lateral lumbar image.

25. A system according to claim 24, wherein a last convolutional layer outputs the visual features.

26. A system according to claim 19, wherein each of the anterior and posterior decoders comprises a trained CNN.

27. A system according to claim 26, wherein each of the anterior and posterior decoders comprises a long-short term memory network and an attention module.

28. A system according to claim 26, wherein each of the anterior and posterior decoders comprises a global pooling layer, a dense layer with rectified linear activation units (Relu activation), and a dense layer with a linear activation.

29. A system according to claim 19, wherein the at least one processor is arranged to implement a risk category classifier for determining a risk category for a disease.

30. (canceled)

31. A method of diagnosing a disease, the method comprising:
receiving a lateral lumbar image that includes an abdominal aortic section of a patient's abdominal aorta, the abdominal aortic section including a plurality of abdominal aortic segments;
determining a score representing abdominal aortic calcification from the lateral lumbar image using one or more processors, wherein the determining step comprises:
the one or more processors encoding the lateral lumbar image to identify visual features in the image at the abdominal aortic segments;
the one or more processors implementing an anterior decoder to decode the visual features to produce a plurality of anterior calcification scores, each anterior calcification score associated with an anterior portion of one abdominal aortic segment;
the one or more processors implementing a posterior decoder to decode the visual features to produce a plurality of posterior calcification scores, each posterior calcification score associated with an posterior portion of one abdominal aortic segment;
wherein each of the anterior and posterior decoders is a trained machine learning decoder and the anterior decoder is trained separately to the posterior decoder; and
the method comprising determining a risk category for a disease based on the determined plurality of anterior and posterior calcification scores.

32. (canceled)

33. A method of diagnosing or prognosing a disease or condition in a human comprising classifying calcification scores into risk categories, the calcification scores obtained by a method of determining abdominal aortic calcification as claimed in claim 1, and identifying a prognosis of the disease from the determined risk category.

34. A method according to claim 33, wherein the disease or condition comprises CVD, diabetes, dementia, or osteoporosis, a risk of later life falls and/or fractures.

35. A program for controlling one or more processors, the program comprising instructions stored in a non-volatile medium to control the one or more processors to implement a method as claimed in claim 1.
